(19) United States
(12) Patent Application Publication
(10) Pub. No.: US 2008/0279930 A1
(43) Pub. Date: Nov. 13, 2008

(54) Controlled-Release Flupirtine Compositions, Compacts, Kits and Methods of Making and Use Thereof

(76) Inventors: Bernd Terhaag, Pirna (DE); Asal Qadan, Dresden (DE); Joachim Wolf, Radebeul (DE); Barbara Faustmann, Dresden (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/745,374

(22) Filed: May 7, 2007

(51) Int. Cl. A61K 9/52; A61K 31/44; A61K 9/02; A61K 9/14; A61P 43/00; A61K 9/26; A61K 9/50

(52) U.S. Cl. 424/456; 424/469; 424/489; 424/490; 424/493; 424/494; 424/497; 514/357

(57) ABSTRACT

The present invention relates to compositions and compacts comprising flupirtine or a pharmaceutically acceptable salt thereof in which there is controlled-release of at least a portion of flupirtine or a pharmaceutically acceptable salt thereof. The invention further relates to kits comprising such compositions and compacts, and methods of making and using such compositions and compacts.

### BACKGROUND OF THE INVENTION

1. Field of the Invention

The present invention relates to compositions and compacts comprising flupirtine or a pharmaceutically acceptable salt thereof in which there is controlled-release of at least a portion of flupirtine or a pharmaceutically acceptable salt thereof. The invention further relates to kits comprising such compositions and compacts, and methods of making and using such compositions and compacts.

2. Background Art

Flupirtine is a centrally-acting, non-opioid analgesic with muscle relaxant and neuroprotective properties (Jakob, R. and Krieglstein, J., Br. J. Pharmacol. 122: 1333-1338 (1997); Osborne, N. N., et al., Gen. Pharmac. 30: 255-263 (1998); Zimmer, G., et al., Br. J. Pharmacol. 123: 1154-1158 (1998)). Flupirtine indirectly acts as a N-methyl-D-aspartate (NMDA)-antagonist, resulting in indirect inhibition of NMDA-induced intracellular influx of Ca⁺⁺. It is the prototype of a class of agents known as Selective NEuronal Potassium Channel Openers (SNEPCO) and activates G-protein-activated inwardly rectifying K⁺ (GIRK) channels of the neuron. As a result of flupirtine administration, K⁺ is discharged, the resting neuronal membrane potential is stabilized, and neuronal membrane activation (excitation) is reduced. Thus, the activation of the NMDA receptor is indirectly antagonized by flupirtine, as the Mg²⁺ block of the NMDA receptor is only neutralized on the depolarization of the cell membrane (Kornhuber, J., et al., Fortschr Neurol Psychiatr 67: 466-475 (1999); Jakob, R. and Krieglstein, J.; Osborne, N. N., et al.; Zimmer, G., et al.).

Flupirtine is rapidly released from standard formulations, such that its analgesic effect sets in quickly and lasts for only a brief duration. Therefore, the treatment of strong chronic pain using typical formulations of flupirtine requires frequent administration of the drug in relatively short intervals in order to ensure a sufficient concentration of flupirtine in the blood plasma of the patient. The necessity of frequent administration can easily lead to mistakes in patient intake as well as undesirable fluctuations in plasma concentration, which is disadvantageous for both patient compliance and therapeutic use, in particular for the treatment of chronic pain. As such, there is a need for a controlled-release formulation of flupirtine that allows for a consistent and lasting release over a longer period, thus requiring less frequent administration.

### BRIEF SUMMARY OF THE INVENTION

The present invention is directed to pharmaceutical compositions comprising compacts comprising flupirtine or a pharmaceutically acceptable salt thereof, wherein each of the compacts has a particle size of about 160 μm to about 800 μm; wherein at least a portion of the compacts are coated with a controlled-release component for controlled-release of flupirtine or a pharmaceutically acceptable salt thereof; and wherein the compacts coated with a controlled-release component are individually coated. The composition can be selected from the group consisting of a tablet, a film tablet, a hard gelatin capsule, a soft gelatin capsule, a pellet, a granulate, a pill, a micro-capsule, and a suppository.

In some embodiments, each of the compacts has a particle size of about 250 μm to about 500 μm.

In some embodiments, each of the compacts is spherical or approximately spherical.

In some embodiments, flupirtine or a pharmaceutically acceptable salt thereof is released at a uniform rate from the compacts coated with a controlled-release component.

In some embodiments, the compacts have a bulk volume selected from the group consisting of less than about 5 ml/g, less than about 3 ml/g, less than about 2.5 ml/g, and from about 0.8 ml/g to about 2.5 ml/g. In some embodiments, the compacts have a bulk volume of less than about 5 ml/g. In some embodiments, the compacts have a bulk volume of less than about 3 ml/g. In some embodiments, the compacts have a bulk volume of less than about 2.5 ml/g. In some embodiments, the compacts have a bulk volume of from about 0.8 ml/g to about 2.5 ml/g.

In some embodiments, the controlled-release component is a polymer film, wherein the polymer film comprises at least one polymer or copolymer. The polymer or copolymer can be selected from the group consisting of acrylic acid, acrylic acid derivatives, methacrylic acid, methacrylic acid derivatives, and combinations thereof. In some embodiments, the ratio of the weight of the polymer film to the weight of flupirtine or a pharmaceutically acceptable salt thereof is selected from the group consisting of from about 0.001 to about 20, from about 0.01 to about 10, and from about 0.05 to about 0.1. In some embodiments, the ratio of the weight of the polymer film to the weight of flupirtine or a pharmaceutically acceptable salt thereof is from about 0.001 to about 20. In some embodiments, the ratio of the weight of the polymer film to the weight of flupirtine or a pharmaceutically acceptable salt thereof is from about 0.01 to about 10. In some embodiments, the ratio of the weight of the polymer film to the weight of flupirtine or a pharmaceutically acceptable salt thereof is from about 0.05 to about 0.1.

In some embodiments, flupirtine or a pharmaceutically acceptable salt thereof is released from the composition in vitro at a rate selected from the group consisting of from about 35 to about 75 percent within 240 minutes, from about 15 to about 35 percent within 15 minutes, from about 55 to about 75 percent within 240 minutes, and from about 75 percent to about 100 percent within 600 minutes. In some embodiments, flupirtine or a pharmaceutically acceptable salt thereof is released from the composition in vitro at a rate of from about 35 to about 75 percent within 240 minutes. In some embodiments, flupirtine or a pharmaceutically acceptable salt thereof is released from the composition in vitro at a rate of from about 15 to about 35 percent within 15 minutes. In some embodiments, flupirtine or a pharmaceutically acceptable salt thereof is released from the composition in vitro at a rate of from about 55 to about 75 percent within 240 minutes. In some embodiments, flupirtine or a pharmaceutically acceptable salt thereof is released from the composition in vitro at a rate of from about 75 percent to about 100 percent within 600 minutes.

In some embodiments, the composition further comprises a portion of the compacts formulated for immediate-release of flupirtine or a pharmaceutically acceptable salt thereof.

In some embodiments, the ratio of flupirtine or a pharmaceutically acceptable salt thereof for controlled-release to flupirtine or a pharmaceutically acceptable salt thereof for immediate-release is from about 1:2 to about 9:1. In some embodiments, the ratio of flupirtine or a pharmaceutically acceptable salt thereof for controlled-release to flupirtine or a pharmaceutically acceptable salt thereof for immediate-release is about 3:1.

In some embodiments, the composition further comprises at least one exterior phase component. The exterior phase component can be selected from the group consisting of crosscarmellose-Na, microcrystalline cellulose, microdispersed silicon dioxide, magnesium stearate, powder cellulose, calcium hydrogen phosphate dihydrate, lactose, mannitol, starch, and combinations thereof.

The present invention is also directed to a compact comprising flupirtine or a pharmaceutically acceptable salt thereof, wherein the compact has a particle size from about 160 μm to about 800 μm. In some embodiments, the compact has a particle size from about 250 μm to about 500 μm.

In some embodiments, the compact is coated with a controlled-release component. In some embodiments, the controlled-release component comprises a polymer film.

In some embodiments, the compact has a bulk volume selected from the group consisting of less than about 5 ml/g, less than about 3 ml/g, less than about 2.5 ml/g, and from about 0.8 ml/g to about 2.5 ml/g. In some embodiments, the compact has a bulk volume of less than about 5 ml/g. In some embodiments, the compact has a bulk volume of less than about 3 ml/g. In some embodiments, the compact has a bulk volume of less than about 2.5 ml/g. In some embodiments, the compact has a bulk volume of from about 0.8 ml/g to about 2.5 ml/g.

In some embodiments, the compact is spherical or approximately spherical.

The present invention is further directed to a kit comprising the pharmaceutical compositions or compacts of the invention. In some embodiments, the pharmaceutical composition or compact is packaged in the form of individual dosage units. The individual dosage unit can be selected from the group consisting of a tablet, a film tablet, a hard gelatin capsule, a soft gelatin capsule, a pellet, a granulate, a pill, a micro-capsule, and a suppository.

In some embodiments, the kit further comprises printed instructions for its use.

In some embodiments, the kit further comprises printed matter describing the use of the composition or compact to treat a condition requiring flupirtine therapy, a pre-recorded media device describing the use of the composition or compact to treat a condition requiring flupirtine therapy, or a planner. In some embodiments, the printed matter is a book, booklet, brochure, or leaflet. In some embodiments, the pre-recorded media device is a DVD, a videotape cassette, a CD-ROM, an audiocassette, or an audio compact disk.

The present invention is further directed to a method of flupirtine therapy, the method comprising administering the pharmaceutical compositions of the present invention to a subject in need thereof. The compositions can be selected from the group consisting of a tablet, a film tablet, a hard gelatin capsule, a soft gelatin capsule, a pellet, a granulate, a pill, a microcapsule, and a suppository.

In some embodiments, the subject is in need of pain relief. In some embodiments, the subject is in need of acute pain relief. In some embodiments, the subject is in need of chronic pain relief. In some embodiments, the subject is in need of relief from muscle-skeletal pain, post-operative pain, pain after an injury, or pain caused by a tumor.

In some embodiments, the subject is in need of relief of symptoms caused by a disease or disorder treatable by flupirtine administration. In some embodiments, the disease or disorder is selected from the group consisting of a neurological disorder, a peripheral disorder, episodic ataxia, epilepsy, neuromyotonia, Parkinson's disease, congenital deafness, long QT syndrome, a potassium channelopathy, chronic pain, acute pain, muscle tenseness, apoptotic neuronal cell death, prion disease, Creutzfeldt-Jakob disease, Alzheimer's disease, Tinnitus, overactive bladder, neuropathic pain, neurodegenerative diseases, diabetic neuropathy, diabetic retinopathy, diabetic maculopathy, maculopathy of genetic origin, ocular apoptosis, glaucoma, fibromyalgia, and Batten disease.

In some embodiments, the subject is a human or a non-human animal. In some embodiments, the subject is a human.

In some embodiments, the subject is a non-human animal.

In some embodiments, the composition is administered orally.

In some embodiments, the composition is administered rectally.

In some embodiments, the composition is administered once or twice daily. In some embodiments, the composition is administered once daily. In some embodiments, the composition is administered twice daily.

The present invention is further directed to a method of preparing compacts comprising flupirtine or a pharmaceutically acceptable salt thereof comprising: (a) compacting flupirtine or a pharmaceutically acceptable salt thereof to produce compacts of flupirtine or a pharmaceutically acceptable salt thereof, and (b) selecting compacts with particle sizes from about 160 μm to about 800 μm.

In some embodiments, the compacts are selected with particle sizes of about 250 μm to about 500 μm.

In some embodiments, the method further comprises: (c) coating at least a portion of the compacts with a controlled-release component, wherein the compacts coated with a controlled-release component are individually coated.

In some embodiments, a separating agent is used to prevent adhesion of compacts.

The present invention is further directed to a method of preparing a pharmaceutical composition, wherein the compacts of the present invention are further processed into a composition selected from the group consisting of a tablet, a film tablet, a hard gelatin capsule, a soft gelatin capsule, a pellet, a granulate, a pill, a microcapsule, and a suppository.

### BRIEF DESCRIPTION OF THE DRAWINGS/FIGURES

FIGS. 1A-1D show the needle-like morphology of flupirtine prior to compacting. Scale bars indicate lengths of 5 μm, 5 μm, 20 μm, and 50 μm in FIGS. 1A-1D, respectively.

FIGS. 2A-2C show the particulate morphology of flupirtine after compacting.

FIG. 2A shows compacted particles prior to separation by particle size. FIG. 2B shows the fraction of compacted particles with individual particle sizes from 200 μm to 400 μm in diameter. FIG. 2C shows the fraction of compacted particles with individual particle sizes above 400 μm in diameter.

FIG. 3 shows a graph depicting the in vivo release of flupirtine in μg/ml per hour over a 48 hour period as measured in blood samples following administration of test (T) formulations with controlled-release portions of flupirtine under fasting conditions (Tfa, •) and under the influence of food (Tfe, ∘), and following administration of a reference (R) non-controlled-release capsule formulation (Rfa, ▪). FIG. 3 also includes a table displaying kinetic parameters (k.P.) associated with the treatments.

### DETAILED DESCRIPTION OF THE INVENTION

The present invention is directed to pharmaceutical compositions and compacts comprising flupirtine or a pharmaceutically acceptable salt thereof, kits, and methods of making and using the same, in which there is controlled-release of at least a portion of flupirtine or a pharmaceutically acceptable salt thereof such that administration is required only once or twice daily. Additionally, the invention is associated with improvements in drug safety due to improved bioavailability, due to less frequent occurrence of side effects, and due to avoidance of risks associated with “dose dumping” in which a large amount of flupirtine is released in a brief period following administration.

The embodiments and definitions described herein are intended to illustrate the invention and are not intended to limit the scope of the invention. The section headings are provided solely for organizational purposes and are not intended to impart any meaning or division to this document unless otherwise specified.

### Definitions

Unless otherwise defined, all technical and scientific terms used herein have the meanings commonly understood by one of ordinary skill in the art to which this invention belongs. In the case of conflict, the present application, including definitions, will control.

Although materials and methods similar to or equivalent to those described herein can be used in the practice or testing of the present invention, suitable materials and methods are described below. The materials, methods, and examples are illustrative only and are not intended to be limiting. Other features and advantages of the invention will be apparent from the detailed description and from the claims.

The methods and techniques of the present invention are generally performed, unless otherwise indicated, according to conventional methods well known in the art and as described in various general or specific references that are cited and discussed throughout the present specification. Standard techniques are used for the preparation, formulation, and delivery of pharmaceuticals as well as in the treatment of patients.

In order to further define this invention, the following terms and definitions are provided.

As used herein, “excipient” refers to a component, or mixture of components, that is used in the formulation of the compositions or compacts of the present invention to give desirable characteristics to the composition or compact. As used herein, the term “pharmaceutically acceptable” refers to those compounds, materials, compositions, compacts, salts, and/or dosage forms which are, within the scope of sound medical judgment, suitable for contact with the tissues of human beings and animals without excessive toxicity, irritation, allergic response, or other problematic complications over the desired duration of treatment commensurate with a reasonable benefit/risk ratio. In some embodiments, the term “pharmaceutically acceptable” means approved by a regulatory agency of the Federal or a state government or listed in the U.S. Pharmacopeia or other generally recognized international pharmacopeia for use in animals, and more particularly in humans. Various pharmaceutically acceptable excipients can be used. In some embodiments, the pharmaceutically acceptable excipient can be, but is not limited to, an alkaline agent, a stabilizer, an adhesion agent, a separating agent, a coating agent, an exterior phase component, a controlled-release component, a solvent, a surfactant, a humectant, a buffering agent, a filler, an emollient, or combinations thereof. Excipients in addition to those discussed herein can include excipients listed in, though not limited to, Remington: The Science and Practice of Pharmacy, 21^{st} ed. (2005). Inclusion of an excipient in a particular classification herein (e.g., “solvent”) is intended to illustrate rather than limit the role of the excipient. A particular excipient can fall within multiple classifications.

As used herein, the term “administering” refers to the introduction of a substance into the body of a subject by any route compatible with the formulation, composition, or compact described. A substance is considered to be “administered” if the substance is introduced into the body of the subject by the subject, or if another person, a machine, or a device introduces the substance into the body of the subject. “Administering,” therefore, includes, e.g., self-administration, administration by others, and indirect administration.

The terms “treat” and “treatment” refer to both therapeutic treatment and prophylactic or preventative measures, wherein the object is to prevent or slow down (lessen) an undesired physiological condition, disorder, or disease, or to obtain beneficial or desired clinical results. For purposes of this invention, beneficial or desired clinical results include, but are not limited to, alleviation of the symptoms or signs associated with a condition, disorder, or disease; diminishment of the extent of a condition, disorder, or disease; stabilization of a condition, disorder, or disease (i.e., where the condition, disorder, or disease is not worsening); delay in onset or progression of the condition, disorder, or disease; amelioration of the condition, disorder, or disease state; remission (whether partial or total and whether detectable or undetectable) of the condition, disorder, or disease; or enhancement or improvement of a condition, disorder, or disease. Treatment includes eliciting a clinically significant response without excessive side effects. Treatment also includes prolonging survival as compared to expected survival if not receiving treatment.

By “subject” or “individual” or “animal” or “patient” or “mammal” is meant any subject, particularly a mammalian subject, for whom diagnosis, prognosis, or therapy is desired. Mammalian subjects include, but are not limited to, humans; domestic animals; farm animals; zoo animals; sport animals; pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject.

As used herein, a “therapeutically effective amount” or “therapeutic dose” refers to an amount effective, at dosages and for periods of time necessary, to achieve a desired therapeutic result. A therapeutic result may be, e.g., lessening of symptoms, prolonged survival, improved mobility, and the like. A therapeutic result need not be a “cure.”

As used herein, a “prophylactically effective amount” or “prophylactic dose” refers to an amount effective, at dosages and for periods of time necessary, to achieve the desired prophylactic result. Typically, since a prophylactic dose is used in subjects prior to or at an earlier stage of disease, a prophylactically effective amount will be less than a therapeutically effective amount for treatment of an advanced stage of disease.

As used herein, the term “tumor” refers, in part, to any undesirable proliferation of cells, including malignant and non-malignant tumors, solid or fluid tumors, carcinomas, myelomas, sarcomas, leukemias, lymphomas, and other cancerous, neoplastic, or tumorigenic diseases.

As used herein, “about” refers to plus or minus 10% of the indicated number. For example, “about 200 μm” indicates a range of 180 μm to 220 μm; “about 10%” indicates a range of 9% to 11%.

### Pharmaceutical Compositions and Compacts

Pharmaceutical compositions of the present invention are directed, in part, to controlled-release of flupirtine or pharmaceutically acceptable salts thereof. Pharmaceutically acceptable salts of flupirtine are those salts that are tolerated in pharmaceutical use in mammals and/or humans. In some embodiments, pharmaceutically acceptable salts of flupirtine are formed with inorganic or organic acids. In some embodiments, pharmaceutically acceptable salts of flupirtine comprise flupirtine hydrochloride, flupirtine maleate, and flupirtine-D-gluconate.

The amounts of flupirtine listed in the present application always refer to the base form of flupirtine. When a flupirtine salt is used, the amounts must be converted according to the increased molar weight of the salt form.

In some embodiments, at least a portion of the flupirtine and/or a pharmaceutically acceptable salt thereof is present in the pharmaceutical composition or compact in a controlled-release form. In some embodiments, the entire portion of flupirtine and/or a pharmaceutically acceptable salt thereof is in a controlled-release form.

In some embodiments, the pharmaceutical composition or compact may comprise active ingredients in addition to flupirtine or a pharmaceutically acceptable salt thereof. In some embodiments, the pharmaceutical composition or compact contains only flupirtine or a pharmaceutically acceptable salt thereof.

In some embodiments, the term “controlled-release of flupirtine or a pharmaceutically acceptable salt thereof” means a maximum in vitro release of 75% of flupirtine or a pharmaceutically acceptable salt thereof from the pharmaceutical composition or compact over at least four hours. The in vitro release of flupirtine or a pharmaceutically acceptable salt thereof from the composition or compact can be measured with the use of the European Pharmacopoeia (Ph. Eur.) Paddle Method at 100 rpm in a buffer (according to Ph. Eur.) with a pH of 6.8 at 37° C. and detection under UV-spectrophotometry.

Flupirtine has a needle-shaped crystalline structure. Due to this crystalline structure flupirtine cannot be coated optimally and regularly with a controlled-release component. In general, the edges and tips of the crystalline needles can only be coated with difficulty and irregularity. Compacting associated with the present invention has been surprisingly found to allow for optimal coating of flupirtine such that a reproducible and homogenous coating is obtained. As such, the controlled-release portion comprising flupirtine or a pharmaceutically acceptable salt thereof is compacted to form a compact of flupirtine or a pharmaceutically acceptable salt thereof. In some embodiments, the compact additionally includes pharmaceutically acceptable excipients. Compacting is also used to improve the physical parameters of flupirtine or a pharmaceutically acceptable salt thereof, such as, e.g., particle size and bulk volume.

During compacting, flupirtine or a pharmaceutically acceptable salt thereof is compacted, if necessary with the addition of pharmaceutically acceptable excipients, in a suitable device. Suitable devices, include, but are not limited to, a roller compactor or a roller press in which compacting occurs in the gap between two smooth or profiled rollers rotating in opposite directions. In some embodiments, the strip of material, the so-called “slug,” created by compacting is crushed and subsequently screened to produce a compact with a defined particle size ranging from about 160 μm to 800 μm. In some embodiments the particle size ranges from about 250 μm to about 500 μm. Particle size is defined in terms of the diameter of the particle. In some embodiments, hydraulic compacting forces are used during compacting with such forces ranging from about 3000 kN/m² to about 9000 kN/m². In some embodiments a roller rotation from about 1 rpm to about 10 rpm is used. In some embodiments, the screw rotation may be varied from about 10 rpm to about 70 rpm. The pressure applied by the rollers during the compacting process has only a negligible influence or no influence at all on the release profile of the controlled-release portion.

When flupirtine or a pharmaceutically acceptable salt thereof is not compacted, it is very voluminous and has high bulk and/or stamping volumes. For example, non-compacted flupirtine maleate has a bulk volume ranging from about 7.5 ml/g to about 10.5 ml/g and a stamping volume from about 4 ml/g to about 5.5 ml/g. Non-compacted particle sizes range from about 5 μm to about 100 μm with a large variation of particle sizes (the particle size can be measured according to the 2.9.12 sieve analysis or alternatively by microscopy according to 2.9.13 in accordance with Ph. Eur. 5). A reproducible coating of non-compacted particles with a controlled-release component is not possible with such a large variation in particle sizes. Moreover, the specific surface area of the non-compacted particles ranges from about 110 m²/l to about 180 m²/l.

Additionally, non-compacted flupirtine has poor flow characteristics and exhibits a strong tendency for bridging. This results in imprecise dosing. Furthermore, due to its structure and brittleness, non-compacted flupirtine is difficult to process. As such, the technical difficulties associated with non-compacted flupirtine negatively affect the reproducibility of pharmaceutical preparations, such as tablets in particular.

In contrast, compacted flupirtine or a pharmaceutically acceptable salt thereof is homogenous and exhibits improved flow characteristics and a reduced bulk volume. Additionally, the distribution of particle sizes and the surface area available for coating are precisely defined for compacted flupirtine or a pharmaceutically acceptable salt thereof, in which the specific surface area is about 10 m²/l to about 25 m²/l. This allows the particles of flupirtine or a pharmaceutically acceptable salt thereof and any associated excipients to be evenly coated with the controlled-release component, resulting in a defined release behavior. This allows for a more precise and reproducible dosing of flupirtine or a pharmaceutically acceptable salt thereof.

In some embodiments, the compacts of the present invention have a bulk volume of less than about 5 ml/g. In some embodiments, the compacts of the present invention have a bulk volume of less than about 3 ml/g. In some embodiments, the compacts of the present invention have a bulk volume of less than about 2.5 ml/g. In some embodiments, the compacts of the present invention have a bulk volume from about 0.8 ml/g to about 2.5 ml/g. The bulk volume of the compacts is essentially less than the bulk volume of non-compacted flupirtine, which ranges approximately from about 7.5 ml/g to about 10.5 ml/g. The compact of flupirtine or a pharmaceutically acceptable salt thereof according to the present invention is therefore less voluminous, has better flow characteristics, can be better processed in a reproducible manner, and can be dosed more precisely.

By compacting flupirtine or a pharmaceutically acceptable salt thereof, and any associated excipients, a fraction of particles having a narrow size range can be achieved, with the particles having a uniform surface area. Further, the reduction of the bulk volume and the improvement of the flowability allow for coating of flupirtine or a pharmaceutically acceptable salt thereof under reproducible conditions.

The homogenous size and surface area of the compacts surprisingly allows for reproducible and uniform coating of flupirtine or a pharmaceutically acceptable salt thereof with controlled-release components. Therefore, a precise dosage and a consistent release of flupirtine or a pharmaceutically acceptable salt thereof can be achieved from the individual compacts. This means that a defined constant level of flupirtine or a pharmaceutically acceptable salt thereof is achieved in the body by the controlled-release of flupirtine or a pharmaceutically acceptable salt thereof from the pharmaceutical composition over an extended period of time of, e.g., 12 to 24 hours after administration. No dosing variations nor “dose dumping” occur, thus a reduction of side effects is achieved in comparison to conventional flupirtine formulations.

In some embodiments, a compact comprising flupirtine or a pharmaceutically acceptable salt thereof has a particle size from about 160 μm to about 800 μm. In some embodiments, a compact comprising flupirtine or a pharmaceutically acceptable salt thereof has a particle size from about 180 μm to about 700 μm. In some embodiments, a compact comprising flupirtine or a pharmaceutically acceptable salt thereof has a particle size from about 200 μm to about 600 μm. In some embodiments, a compact comprising flupirtine or a pharmaceutically acceptable salt thereof has a particle size from about 250 μm to about 500 μm. In some embodiments, a compact comprising flupirtine or a pharmaceutically acceptable salt thereof is spherical or approximately spherical. The term “compact of flupirtine or a pharmaceutically acceptable salt thereof” includes both singular and plural compacts, so that several compacts comprising flupirtine or a pharmaceutically acceptable salt thereof are also included. In some embodiments, compacts comprising flupirtine or a pharmaceutically acceptable salt thereof are used to produce the pharmaceutical preparations of the present invention.

In some embodiments, the compact comprising flupirtine or a pharmaceutically acceptable salt thereof is coated with a controlled-release component. In some embodiments, the compact comprising flupirtine or a pharmaceutically acceptable salt thereof is coated with a controlled-release component and an additional excipient.

In some embodiments, the controlled-release component comprises a retarding polymer film. In further embodiments, the controlled-release component comprises a retarding polymer film and an additional excipient. In still further embodiments, the additional excipient is a parting agent. In other embodiments, the additional excipient is a pigment. In some embodiments, the retarding polymer film comprises at least one polymer or copolymer, such as, but not limited to acrylic acid, acrylic acid derivatives, methacrylic acid, methacrylic acid derivatives, and combinations thereof. In some embodiments, the polymer film includes, but is not limited to: methacrylic acid and methacrylic acid esters, such as, but not limited to, Eudragit® L and Eudragit® S; a copolymer of acrylic and methacrylic acid esters with a small amount of trimethyl ammonium methacrylate such as Eudragit® RL or Eudragit® RS; a copolymer of acrylic acid and methacrylic acid, as well as their esters (ratio of free carboxylic groups to ester groups, e.g., 1:1), such as Eudragit® L30D; or a copolymer made from acrylic acid ethyl and methacrylic acid methyl ester such as Eudragit® NE30D; or combinations thereof. By using these polymers as controlled-release components, a homogenous and safe release rate is achieved.

In some embodiments, the controlled-release component is a swellable, but water-insoluble, ethylacrylate/methylmethacrylate copolymer with neutral ester groups, such as Eudragit® NE30D.

When coating the compacted flupirtine or a pharmaceutically acceptable salt thereof with a controlled-release component, additional pharmaceutically acceptable excipients may be used that do not influence the release of flupirtine or a pharmaceutically acceptable salt thereof, such as parting agents or pigments. Because some polymer films with low transfer temperatures, such as, e.g., Eudragit® NE30D, have adhesive characteristics, compacts can adhere to each other when coated with such films, for example, in a fluidized bed. Repeated adhesion and separation of the compact during coating leads to a faulty positioning in the bed. This can be prevented by the use of parting agents. Use of a parting agent with adherent films can therefore allow for a reproducible and controlled-release of flupirtine or a pharmaceutically acceptable salt thereof and thus prevent so-called “dose dumping” due to flawed coating In some embodiments, the parting agent includes, but is not limited to, talcum, magnesium stearate, glycerol monostearate, calcium arachinate, glycerol palmitostearate, stearinic acid, and triglycerides.

The controlled-release component ensures a diffusion-controlled-release of flupirtine or a pharmaceutically acceptable salt thereof. The release is therefore pH- and matrix-independent. As soon as liquid diffuses through a coating of the controlled-release component, the coating swells and becomes more permeable. Flupirtine or a pharmaceutically acceptable salt thereof then comes into contact with the liquid and dissolves, to be released by diffusion through the coating. The release of flupirtine or a pharmaceutically acceptable salt thereof thus occurs by way of diffusion from the individually coated compacts. Due to the fact that the release is controlled by the diffusion from the compacts that are individually coated with a controlled-release component, a particularly consistent, continuous release of flupirtine or a pharmaceutically acceptable salt thereof is ensured over an extended period of time, in contrast to pH- and matrix-dependent controlled-release formulations. This results in a safe, therapeutically effective dosage at less frequent intervals.

Advantageously, the addition of excipients such as softeners, tensides, anti-foaming agents, emulsifiers, and pore formers can be omitted from pharmaceutical compositions and compacts of the present invention. Even without the addition of external softeners, such as, e.g., glycerol triacetate, tributyl citrate, dibutyl sebacate, and dibutyl phthalate, the coating is sufficiently elastic to resist mechanical forces occurring, for example, during the subsequent tablet formation. Surprisingly, a coating can be produced without any or with a tolerable number of flaws, and reproducible release rates can be achieved, in spite of the fact that the use of pore formers is omitted. When used, pore formers are usually distributed homogenously in the coating and are dissolved out of the film coating in the releasing medium, resulting in the formation of pores through which liquid from the releasing medium penetrates the generally impermeable film coating. In some embodiments, pore formers are omitted from the controlled-release component. In some embodiments, the controlled-release component additionally comprises a pore former, such as, e.g., alkali salt, polyvalent alcohols, saccharose, mannite, sorbite, soluble polymers such as carbopol, and polyethylene glycol.

Coating with the controlled-release component occurs through common methods known in the art, for example, by spraying with a solution of the controlled-release component containing organic solvents or suspensions of the controlled-release component in organic solvents or water. The spraying occurs using, e.g., the air suspension method (e.g., Glatt fluidized bed WSG.)

In some embodiments, the ratio of the weight of the controlled-release polymer film to flupirtine or a pharmaceutically acceptable salt thereof ranges from about 0.001 to about 20. In some embodiments, the ratio of the weight of the controlled-release polymer film to flupirtine or a pharmaceutically acceptable salt thereof ranges from about 0.01 to about 10. In some embodiments, the ratio of the weight of the controlled-release polymer film to flupirtine or a pharmaceutically acceptable salt thereof ranges from about 0.05 to about 0.1. In such embodiments, an economical production of pharmaceutical compositions in the form of so-called “multiple unit dosage forms” with a controlled-release component is yielded in spite of only small amounts of a controlled-release polymer film.

In the present invention, release of flupirtine or a pharmaceutically acceptable salt thereof occurs from individual compacts. As such, the pharmaceutical compositions of the present invention comprising such compacts belong to the group of dosage forms described as “multiple unit dosage forms” and not to the group described as “single unit dosage forms.” Additionally, in the present invention, controlled-release compacts are individually coated with a controlled-release component. Because of the latter, it is now possible according to the invention to provide flupirtine in dividable drug preparations. For example, a tablet comprising flupirtine can be produced, which can arbitrarily be divided without destroying the controlled-release layer associated with a compact and without influencing the release profile. This also prevents “dose-dumping.”

The compacting of flupirtine or a pharmaceutically acceptable salt thereof can occur with or without the addition of excipients. The substances used as excipients for compacting flupirtine or a pharmaceutically acceptable salt thereof can be provided with certain physical-chemical characteristics, e.g., high plasticity, acceptable flowability, and good compressibility. Further, they do not generally react with other substances and are not toxic. For example, micro-crystalline cellulose may be used as an excipient for compacting.

By adjusting the above-mentioned process parameters during compacting, even without the addition of excipients, spherical or approximately spherical compacts of flupirtine or a pharmaceutically acceptable salt thereof with a narrow distribution of particle sizes and a defined surface area can be obtained, which ensures a reproducible coating with the controlled-release component. Thus, an even and controlled-release of flupirtine or a pharmaceutically acceptable salt thereof is possible when compacted without the addition of excipients, with avoidance of so-called “dose-dumping.”

The pharmaceutical compositions according to the present invention can be taken by the patient once or twice per day due to the delayed release of flupirtine or a pharmaceutically acceptable salt thereof. In some embodiments, the pharmaceutical compositions of the present invention are administered once per day. In some embodiments, the pharmaceutical compositions of the present invention are administered once every 24 hours. In some embodiments, the pharmaceutical compositions of the present invention are administered twice per day. In further embodiments, the pharmaceutical compositions of the present invention are administered twice per day at intervals of 12 hours, in order to ensure a sufficient plasma concentration of flupirtine or a pharmaceutically acceptable salt thereof.

Due to less frequent dosing, patient compliance with treatment regimens can be considerably improved. In particular, patients with strong pain, for example patients with chronic back pain or patients with tumor pain, consider less frequent drug administration and therapeutic effects of longer duration to be advantageous, in particular for night-time pain.

Further, the composition according to the invention is better tolerated than conventional, rapidly releasing formulations with the same concentration of flupirtine. Side effects associated with typical flupirtine formulations, such as fatigue and nausea, occur considerably less frequently and with decreased intensity using the pharmaceutical compositions of the present invention.

In the “multiple unit dosage form” according to the present invention, the release of flupirtine or a pharmaceutically acceptable salt thereof occurs largely independent from the type and amount of food consumed by the patient. Even after food consumption, a consistent release of flupirtine or a pharmaceutically acceptable salt thereof is achieved. Any pharmaceutically acceptable route for the administration of the pharmaceutical compositions of the present invention may be chosen. In some embodiments, the pharmaceutical compositions of the present invention are administered orally. In some embodiments, the pharmaceutical compositions of the present invention are administered rectally.

Also extraordinarily surprising is the observation that when administering the controlled-release pharmaceutical compositions of the present invention to human subjects, an only slightly reduced bioavailability can be achieved in comparison to typical formulations of immediate-release flupirtine.

In some embodiments, flupirtine or a pharmaceutically acceptable salt thereof is released from a pharmaceutical composition of the present invention in vitro at a rate of from about 35 to about 75 percent within 240 minutes. In some embodiments, flupirtine or a pharmaceutically acceptable salt thereof is released from a pharmaceutical composition of the present invention in vitro at a rate of from about 15 to about 35 percent within 15 minutes. In some embodiments, flupirtine or a pharmaceutically acceptable salt thereof is released from a pharmaceutical composition of the present invention in vitro at a rate of from about 55 to about 75 percent within 240 minutes. In some embodiments, flupirtine or a pharmaceutically acceptable salt thereof is released from a pharmaceutical composition of the present invention in vitro at a rate of about 75 percent within 600 minutes. In these embodiments, a long-lasting and consistent release of flupirtine or a pharmaceutically acceptable salt thereof is achieved, allowing administration of the composition once or twice per day.

The in vitro rate of release of flupirtine or a pharmaceutically acceptable salt thereof from the pharmaceutical composition is measured with the use of Ph. Eur. Paddle Method at 100 rpm in a buffer (according to Ph. Eur.) at a pH of 6.8 at 37° C. and with UV-spectrophotometric detection.

In some embodiments, the pharmaceutical compositions of the invention comprise a portion of flupirtine or a pharmaceutically acceptable salt thereof formulated for immediate-release in addition to the controlled-release portion. The portion of flupirtine or a pharmaceutically acceptable salt thereof for immediate-release, e.g., a portion without any controlled-release coating, can be provided compacted or non-compacted or as a combination of both forms. In some embodiments, “immediate-release of flupirtine or a pharmaceutically acceptable salt thereof” means an in vitro release of at least 80% of flupirtine or a pharmaceutically acceptable salt thereof within 45 minutes. In some embodiments, “immediate-release of flupirtine or a pharmaceutically acceptable salt thereof” means an in vitro release of at least 80% of flupirtine or a pharmaceutically acceptable salt thereof within 30 minutes. This in vitro rate of release of flupirtine or a pharmaceutically acceptable salt thereof is measured using Ph. Eur. Paddle Method at 75 rpm in 0.1 N hydrochloric acid at 37° C. and with UV-spectrophotometric detection.

In some embodiments, an immediate-release portion of the pharmaceutical composition provides an initial rapid release of flupirtine or a pharmaceutically acceptable salt thereof and ensures a rapid influx of flupirtine or a pharmaceutically acceptable salt thereof into the plasma, which leads to rapid pain reduction in subjects (“rapid onset”). In these embodiments, the controlled-release portion additionally provides a long-lasting pain-killing effect over a relatively long period (approximately 12 to 24 hours). In such embodiments, a single pharmaceutical composition achieves both rapid-onset and long-lasting effects.

In some embodiments, the ratio of flupirtine or a pharmaceutically acceptable salt thereof for controlled-release to flupirtine or a pharmaceutically acceptable salt thereof for immediate-release is from about 1:2 to about 9:1. In some embodiments, the ratio of flupirtine or a pharmaceutically acceptable salt thereof for controlled-release to flupirtine or a pharmaceutically acceptable salt thereof for immediate-release is about 3:1. The precise ratio for optimum release rates depends on the dosage strength and the desired release and can be determined through common practices in the pharmaceutical arts.

In some embodiments, the pharmaceutical composition is provided in the form of tablets, film tablets, hard gelatin capsules, soft gelatin capsules, pellets, granulates, pills, microcapsules, or suppositories. In some embodiments, the pharmaceutical composition is administered in the form of tablets. In some embodiments, oblong tablets allow the tablet to be divided for easier swallowing, wherein division of the tablet does not influence the release profile.

In some embodiments, the amount of flupirtine in the pharmaceutical composition is from about 10 mg to about 1000 mg. In some embodiments, the amount of flupirtine in the pharmaceutical composition is from about 50 mg to about 500 mg. In some embodiments, the amount of flupirtine in the pharmaceutical composition is from about 100 mg to about 400 mg. In some embodiments, the amount of flupirtine in the pharmaceutical composition is about 400 mg. In some embodiments, the total amount of flupirtine in the pharmaceutical composition is the total of a desired ratio of immediate-release and controlled-release portions. In some embodiments, the amount of flupirtine in the immediate-release portion is 100 mg and the amount of flupirtine in the controlled-release portion is 300 mg.

In some embodiments, the pharmaceutical composition comprises at least one exterior phase component. Depending on the form of administration, an exterior phase component is, e.g., an embedding means, a parting means, a flow regulation means, and/or lubricants or mixtures thereof. In some embodiments, the exterior phase component includes, but is not limited to, crosscarmellose-Na, microcrystalline cellulose, microdispersed silicon dioxide, magnesium stearate, powdered cellulose, calcium hydrogen phosphate dihydrate, lactose, mannitol, starch, and combinations thereof. In some embodiments, the exterior phase components are selected based on lack of influence upon the release rate of flupirtine or a pharmaceutically acceptable salt thereof.

Due to their plastic deformability and high porosity, embedding agents such as microcrystalline cellulose are able to protect the controlled-release formulations from excessively strong mechanical stress, and are able to buffer the forces acting upon them during production of the compositions. In particular, compression into tablets represents a considerable mechanical stress. The release behavior of the composition can be lost if, for example, during production of the tablet the controlled-release coating is damaged. In order to avoid this, in some embodiments, embedding agents are added during tablet production.

In some embodiments, the pharmaceutical composition comprises an immediate-release portion and a controlled-release portion homogenously mixed under the addition of exterior phase components in order to modify dosage without changing the release of flupirtine or a pharmaceutically acceptable salt thereof.

### Methods of Using the Pharmaceutical Compositions and Compacts

Flupirtine is not addictive, and tolerance does not result from its use. Based on its indirect antagonism of NMDA receptors, flupirtine displays an analgesic effect and inhibits arising nociceptive impulses. It is also effective in the treatment of chronic pain, such as, for example, muscle-skeletal pain, post-operative pain, pain after injuries, or pain caused by tumors. Flupirtine has muscle-relaxant properties associated with inhibited stimulation of motor neurons and corresponding effects on interneurons. Rather than a general muscle-relaxing effect (muscle atony) the effect is primarily a relaxing effect on muscle tenseness such as muscle tenseness of the postural and skeletal muscles. Flupirtine has also been shown to protect against ischemic-induced insults to the retina and brain, to counteract the production of reactive oxygen species caused by ascorbate/iron, and to prevent apoptosis induced by oxidative stress in cells lacking NMDA receptors. In therapeutically relevant concentrations, flupirtine does not bond to α₁₋, α₂₋, 5HT₁₋, 5HT₂₋, dopamine, benzodiazepine, opiate, central muscarinergic or nicotinergic receptors. Approximately 75% of the administered flupirtine dose is metabolized in the liver. The metabolite 2-amino-3-acetamino-6-(4-fluor)-benzyl-aminopyridine is produced by means of hydrolysis of the carbamate structure and acetylization of the produced amine. This metabolite possesses about 25% of the analgesic effect of flupirtine and thus is involved in the effect of flupirtine.

If, as a consequence of stimulation of NMDA receptors, potassium ions enter the cell, the pain stimuli are transmitted to the central nervous system. If corresponding stimuli are maintained for a prolonged time period, the synaptic transmission is amplified through a process called “long term potentiation,” resulting in pain chronification. Chronification processes are neuronal conduction processes caused by the plasticity of neuronal functions. The plasticity of neuronal functions allows for a mechanism called “wind up” in which subsequently incoming impulses are amplified. Indirect antagonism of NMDA receptors by flupirtine results in counteraction of chronification and/or, in an existing chronification, a “deletion” of the pain memory, due to the stabilization of neuronal membrane potentials. As such, one object of the invention is the method of treating pain chronification by administration of the pharmaceutical compositions of the present invention.

In some embodiments, the present invention is directed to methods of flupirtine therapy comprising administering the pharmaceutical compositions or compacts described herein to a subject in need thereof. In some embodiments, the subject is in need of pain relief. In some embodiments, the subject is in need of acute pain relief. In some embodiments, the subject is in need of chronic pain relief. In some embodiments, the subject is in need of relief from muscle-skeletal pain, post-operative pain, pain after injury, or pain caused by a tumor.

In some embodiments, the subject is in need of relief of symptoms caused by a disease or disorder treatable by flupirtine administration. In some embodiments, the disease or disorder includes, but is not limited to, a neurological disorder, a peripheral disorder, episodic ataxia, epilepsy, neuromyotonia, Parkinson's disease, congenital deafness, long QT syndrome, a potassium channelopathy, chronic pain, acute pain, muscle tenseness, apoptotic neuronal cell death, prion disease, Creutzfeldt-Jakob disease, Alzheimer's disease, Tinnitus, overactive bladder, neuropathic pain, neurodegenerative diseases, diabetic neuropathy, diabetic retinopathy, diabetic maculopathy, maculopathy of genetic origin, ocular apoptosis, glaucoma, fibromyalgia, and Batten disease.

In some embodiments, the subject is a non-human animal. In some embodiments, the subject is a human. In some embodiments, the pharmaceutical composition or compact is administered orally. In some embodiments, the pharmaceutical composition or compact is administered rectally.

In some embodiments, the present invention is directed to a method for producing compacts comprising flupirtine or a pharmaceutically acceptable salt thereof comprising: (a) compacting flupirtine or a pharmaceutically acceptable salt thereof to produce compacts of flupirtine or a pharmaceutically acceptable salt thereof, and (b) selecting compacts with particle sizes from about 160 μm to about 800 μm. In further embodiments, compacts are selected with particle sizes from about 250 μm to about 500 μm. In some embodiments, the method further comprises: (c) coating at least a portion of the compacts with a controlled-release component, wherein the compacts coated with a controlled-release component are individually coated.

### Kits Comprising the Pharmaceutical Compositions or Compacts

In addition to any kit mentioned previously, the invention also provides kits comprising the pharmaceutical compositions or compacts of the present invention. These kits can include one or more containers filled with one or more of the ingredients of the pharmaceutical compositions or compacts of the invention.

In some embodiments, the kit comprises a container for the pharmaceutical composition or compact of the present invention. Suitable containers include, for example, a bottle, a box, a blister card, a foil packet, or a combination thereof. Optionally, the kit also contains directions for properly administering the pharmaceutical compositions or compacts. The kits can also be designed in a manner such that they are tamper resistant or designed to indicate if tampering has occurred. Optionally, the kit of the present invention can contain the pharmaceutical compositions or compacts of the present invention in combination with other pharmaceutical compositions. In some embodiments, the pharmaceutical composition or compact is an individual dosage unit. In further embodiments, the individual dosage unit includes, but is not limited to, a tablet, a film tablet, a hard gelatin capsule, a soft gelatin capsule, a pellet, a granulate, a pill, a micro-capsule, and a suppository.

Optionally associated with the container(s) in the kits of the present invention can be a notice or printed instructions. Such printed instructions can be in a form prescribed by a governmental agency regulating the manufacture, use, or sale of pharmaceuticals or biological products, which notice reflects approval by the agency of the manufacture, use, or sale for human administration to treat a condition that could be treated by flupirtine therapy. In some embodiments, the kit further comprises printed matter, which, e.g., provides information on the use of the pharmaceutical compositions or compacts to treat a condition or disease or a pre-recorded media device which, e.g., provides information on the use of the pharmaceutical compositions or compacts to treat a condition or disease, or a planner.

“Printed matter” can be, for example, one of a book, booklet, brochure or leaflet. The printed matter can describe the use of the pharmaceutical compositions or compacts of the present invention to treat a condition or disease. Possible formats include, but are not limited to, a bullet point list, a list of frequently asked questions (FAQ) or a chart. Additionally, the information to be imparted can be illustrated in non-textual terms using pictures, graphics, or other symbols.

“Pre-recorded media device” can be, for example, a visual media device, such as a videotape cassette, a DVD (digital video disk), filmstrip, 35 mm movie, or any other visual media device. Alternately, pre-recorded media device can be an interactive software application, such as a CD-ROM (compact disk-read only memory) or floppy disk. Alternately, pre-recorded media device can be, for example, an audio media device, such as a record, audiocassette, or audio compact disk. The information contained on the pre-recorded media device can describe the use of the pharmaceutical compositions or compacts of the present invention to treat a condition or disease.

A “planner” can be, for example, a weekly, a monthly, a multi-monthly, a yearly, or a multi-yearly planner. The planner can be used as a diary to monitor dosage amounts, to keep track of dosages administered, or to prepare for future events wherein taking a regularly administered pharmaceutical compositions or compacts of the present invention can be difficult. Alternately, the planner can be a calendar which will provide a means to monitor when a dosage has been taken and when it has not been taken. This type of planner will be particularly useful for patients having unusual schedules for administering medication to themselves. Additionally, the planner can be useful for the elderly, children, or other patient group who may administer medication to themselves and may become forgetful. One skilled in the art will appreciate the variety of planning tools that would be appropriate for use with the present invention.

The kit can also include a container for storing the other components of the kit. The container can be, for example, a bag, box, envelope or any other container that would be suitable for use in the present invention. Preferably, the container is large enough to accommodate each component and/or any administrative devices that may be accompany the pharmaceutical compositions or compacts of the present invention. However, in some cases, it may be desirable to have a smaller container which can be hidden in a patient's pocketbook, briefcase, or pocket.

In some embodiments, the present invention includes a kit comprising an oral pharmaceutical composition or compact of the present invention. In some embodiments, the present invention includes a kit comprising a rectal pharmaceutical composition or compact of the present invention. In some embodiments, the kit further comprises printed instructions for its use. In some embodiments, the kit further comprises a printed matter, a pre-recorded media device, or a planner describing the use of the pharmaceutical compositions or compacts of the present invention to treat or prevent a condition which could be aided by flupirtine therapy.

In some aspects, the present invention provides a method of delivering the pharmaceutical compositions or compacts of the present invention, to a patient in need thereof, the method comprising: (a) registering in a computer readable storage medium the identity of a physician permitted to prescribe the pharmaceutical compositions or compacts; (b) providing the patient with counseling information concerning a risk attendant to the pharmaceutical compositions or compacts; (c) obtaining informed consent of the patient to receive the pharmaceutical compositions or compacts despite the risk; (d) registering the patient in the computer readable medium after obtaining the informed consent; and (e) permitting the patient access to the pharmaceutical compositions or compacts.

In some embodiments of this method, the access to the pharmaceutical compositions or compacts form is a prescription.

Still other aspects of the present invention include a method of educating a consumer regarding the pharmaceutical compositions or compacts of the present invention, the method comprising distributing the pharmaceutical compositions or compacts to a consumer with consumer information at a point of sale.

In some embodiments, the consumer information is presented in a format including, but not limited to: English language text, a foreign language text, a visual image, a chart, a telephone recording, a website, and access to a live customer service representative. In some embodiments, the consumer information is a direction for use, appropriate age use, indication, contraindication, appropriate dosing, warning, telephone number, or website address.

In some embodiments, the method of educating the consumer further comprises providing professional information to a relevant person in a position to answer a consumer question regarding the pharmaceutical compositions or compacts. In some embodiments, the relevant person is a physician, physician assistant, nurse practitioner, pharmacist, or customer service representative.

In some embodiments, the distributing of the pharmaceutical compositions or compacts is to a location with a pharmacist or a health care provider.

Having generally described this invention, a further understanding can be obtained by reference to the examples provided herein. These examples are for purposes of illustration only and are not intended to be limiting.

### EXAMPLES

### Example 1

### Morphology of Flupirtine

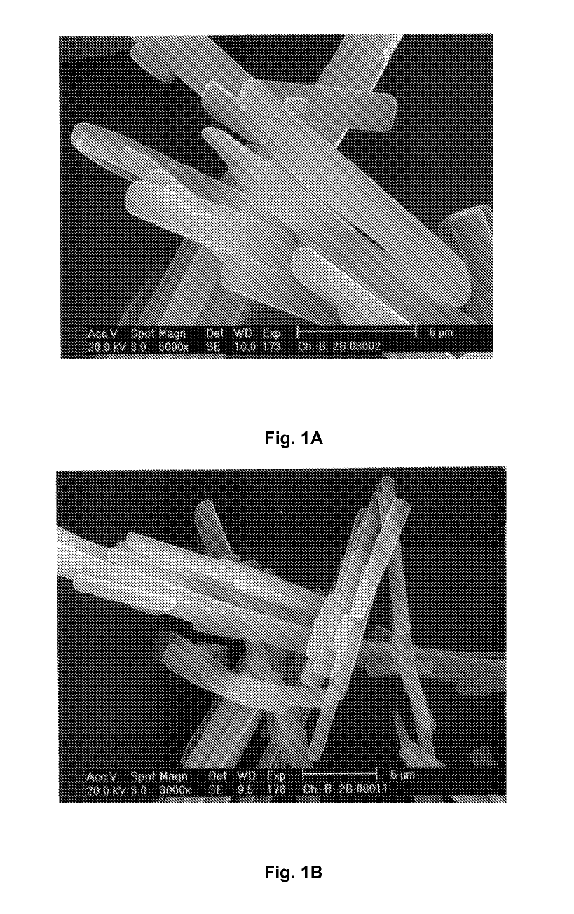

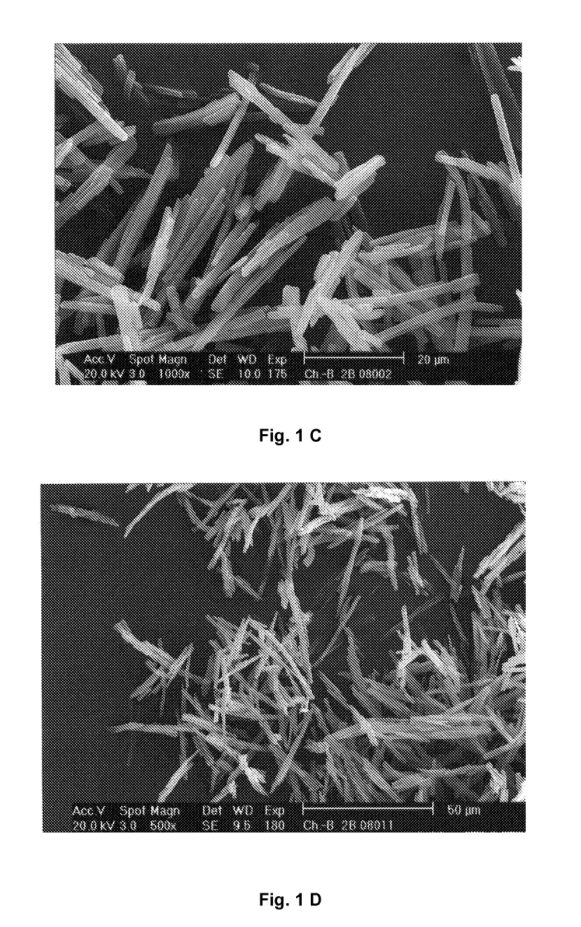

Flupirtine maleate crystals are shown and in FIGS. 1A-1D. The needle-shaped morphology is clearly discernible.

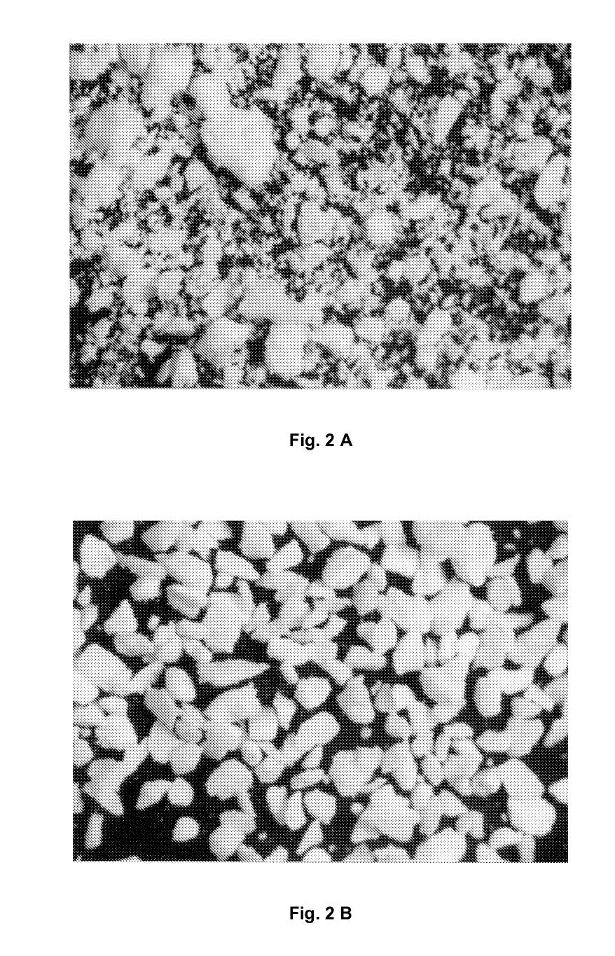

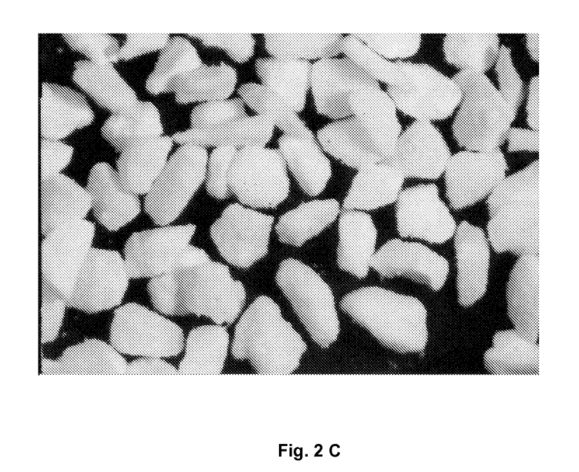

FIG. 2 shows the morphology of flupirtine maleate crystals after compacting (compactor Alexanderwerk). FIG. 2A shows the compacts without any separation. FIG. 2B shows compacts selected for particle sizes of 200-400 μm in diameter. FIG. 2C shows compacts selected for particle sizes above 400 μm in diameter.

It is evident from the Figures that the morphology of flupirtine only allows for homogenous and reproducible coating with a controlled-release component after compacting and separation or fractioning.

### Example 2

### Controlled-Release Tablet Comprising Flupirtine Maleate

Flupirtine maleate was compacted in a dry granulator (roller press), broken, and subsequently fractioned into a grain size from about 250 μm to about 500 μm via a classification sieving machine. 13 kg compacted flupirtine maleate was coated in a fluidized bed granulator with 3.25 kg of an aqueous suspension of poly(ethylacrylate methyl methacrylate)-dispersion, 30% Eudragit® NE 30 D, 0.975 kg talcum, and 0.098 kg of the yellow pigment ferrous oxide. The granulate was then dehydrated, with the product temperature amounting to no more than 35° C. This granulate was mixed with 0.063 kg microdispersed silicon dioxide in the fluidized bed granulator and subsequently sieved via a pendulum sieving machine (mesh size 1 mm).

### Example 3

### Tablet Comprising a Portion of Immediate-Release Flupirtine Maleate and a Portion of Controlled-Release Flupirtine Maleate

For producing 13.896 kg granulate with an immediate-release of flupirtine maleate, 7.2 kg flupirtine maleate was pre-mixed with 2.736 kg calcium hydrogen phosphate-dihydrate, 0.36 kg crosscarmellose-Na, 2.376 kg microcrystalline cellulose, and 0.288 kg of the yellow pigment ferrous oxide in a diffusion mixer. The mixture was then granulated in a high-shear granulator with 0.576 kg methylhydroxypropylcellulose in purified water, and subsequently dried in a fluidized bed dryer at a production temperature not exceeding 35° C. The dried granulate of immediate-release flupirtine maleate was then sieved by a pendulum sieving machine (mesh size: 1 mm).

15 kg of controlled-release granulate produced according to Example 2 and 8.302 kg granulate with immediate-release flupirtine maleate were homogenized with 1.288 kg crosscarmellose-Na, 0.935 kg microcrystalline cellulose, 0.026 kg microdispersed silicon dioxide, and 0.258 kg magnesium stearate in a diffusion mixer.

Using a rotary pelleting press, the mixture was then pressed into convex, oblong tablets with a length of 16 mm and a width of 7 mm, with a dividing notch on one side for dividing the tablet.

The tablets were subjected to in vitro and in vivo testing. In a 3-fold crossover examination of 24 healthy test subjects, the kinetic profile and the bioavailability of the tablets (“Test”) were compared to a conventional capsule formulation with non-controlled-release of flupirtine (“Reference”) after a single administration. Under fasting conditions, subjects were administered either four capsules of the Reference formulation (for a total of 400 mg flupirtine maleate) or a tablet of the Test formulation (for a total of 400 mg flupirtine maleate in a ratio of 100 mg of immediate-release flupirtine and 300 mg controlled-release flupirtine) and the plasma concentration curve was determined over 48 hours for the Test formulation (Tfa) or 36 hours for the Reference formulation (Rfa). Furthermore, the influence of food on the kinetics of the Test formulation (Tfe) was examined over 48 hrs.

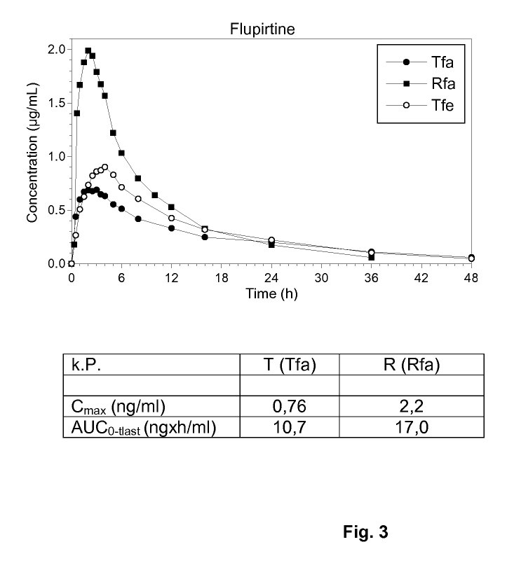

The plasma concentration curves of flupirtine yielded are displayed in FIG. 3. As expected, the maximum concentration (Cₘₐₓ) of the “Test”-formulation under fasting conditions (Tfa) amounted to 25% of the maximum concentration of the Reference capsule formulation (Rfa) based on the rapid absorption of the portion of the tablet with immediate-release of flupirtine (see the table in FIG. 3, with k.P. meaning “kinetic parameter,” T “test,” and R “reference”).

The bioavailability of flupirtine (determined by the area below the blood level curve within the observation period [AUG₀₋ₜₗₐₛₜ]) amounted to 63% (range 59-67%). However, a therapeutically relevant blood level was shown over 24 hours. The curve of the flupirtine concentration under the influence of food (Tfe) was slightly higher than under fasting conditions (Tfa). However, this is not due to changed galenics but is a known feature of the substance.

The relative frequency of the specified side-effects was considerably lower for the Test composition at 8% (=10 side-effects) compared to the Reference capsule formulation at 75% (=89 side-effects) and confirms that a reduction in undesired effects occurs when the rate at which flupirtine enters the systemic circulation is changed.

These examples illustrate possible methods of the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A pharmaceutical composition comprising compacts comprising flupirtine or a pharmaceutically acceptable salt thereof,
wherein each of the compacts has a particle size of about 160 μm to about 800 μm;
wherein at least a portion of the compacts are coated with a controlled-release component for controlled-release of flupirtine or a pharmaceutically acceptable salt thereof; and
wherein the compacts coated with a controlled-release component are individually coated.

2. The pharmaceutical composition of claim 1, wherein each of the compacts has a particle size of about 250 μm to about 500 μm.

3. The pharmaceutical composition of any one of claims 1 or 2, wherein each of the compacts is spherical or approximately spherical.

4. The pharmaceutical composition of any one of claims 1 to 3, wherein flupirtine or a pharmaceutically acceptable salt thereof is released at a uniform rate from the compacts coated with a controlled-release component.

5. The pharmaceutical composition of any one of claims 1 to 4, wherein the compacts have a bulk volume selected from the group consisting of less than about 5 ml/g, less than about 3 ml/g, less than about 2.5 ml/g, and from about 0.8 ml/g to about 2.5 ml/g.

6. The pharmaceutical composition of claim 5, wherein the compacts have a bulk volume of less than about 5 ml/g.

7. The pharmaceutical composition of claim 5, wherein the compacts have a bulk volume of less than about 3 ml/g.

8. The pharmaceutical composition of claim 5, wherein the compacts have a bulk volume of less than about 2.5 ml/g.

9. The pharmaceutical composition of claim 5, wherein the compacts have a bulk volume of from about 0.8 ml/g to about 2.5 ml/g.

10. The pharmaceutical composition of any one of claims 1 to 9, wherein the controlled-release component is a polymer film, wherein the polymer film comprises at least one polymer or copolymer selected from the group consisting of acrylic acid, acrylic acid derivatives, methacrylic acid, methacrylic acid derivatives, and combinations thereof.

11. The pharmaceutical composition of claim 10, wherein the ratio of the weight of the polymer film to the weight of flupirtine or a pharmaceutically acceptable salt thereof is selected from the group consisting of from about 0.001 to about 20, from about 0.01 to about 10, and from about 0.05 to about 0.1.

12. The pharmaceutical composition of claim 11, wherein the ratio of the weight of the polymer film to the weight of flupirtine or a pharmaceutically acceptable salt thereof is from about 0.001 to about 20.

13. The pharmaceutical composition of claim 11, wherein the ratio of the weight of the polymer film to the weight of flupirtine or a pharmaceutically acceptable salt thereof is from about 0.01 to about 10.

14. The pharmaceutical composition of claim 11, wherein the ratio of the weight of the polymer film to the weight of flupirtine or a pharmaceutically acceptable salt thereof is from about 0.05 to about 0.1.

15. The pharmaceutical composition of any one of claims 1 to 14, wherein flupirtine or a pharmaceutically acceptable salt thereof is released from the composition in vitro at a rate selected from the group consisting of from about 35 to about 75 percent within 240 minutes, from about 15 to about 35 percent within 15 minutes, from about 55 to about 75 percent within 240 minutes, and from about 75 percent to about 100 percent within 600 minutes.

16. The pharmaceutical composition of claim 15, wherein flupirtine or a pharmaceutically acceptable salt thereof is released from the composition in vitro at a rate of from about 35 to about 75 percent within 240 minutes.

17. The pharmaceutical composition of claim 15, wherein flupirtine or a pharmaceutically acceptable salt thereof is released from the composition in vitro at a rate of from about 15 to about 35 percent within 15 minutes

18. The pharmaceutical composition of claim 15, wherein flupirtine or a pharmaceutically acceptable salt thereof is released from the composition in vitro at a rate of from about 55 to about 75 percent within 240 minutes.

19. The pharmaceutical composition of claim 15, wherein flupirtine or a pharmaceutically acceptable salt thereof is released from the composition in vitro at a rate of from about 75 percent to about 100 percent within 600 minutes.

20. The pharmaceutical composition of any one of claims 1 to 19, wherein the composition further comprises a portion of the compacts formulated for immediate-release of flupirtine or a pharmaceutically acceptable salt thereof.

21. The pharmaceutical composition of claim 20, wherein the ratio of flupirtine or a pharmaceutically acceptable salt thereof for controlled-release to flupirtine or a pharmaceutically acceptable salt thereof for immediate-release is from about 1:2 to about 9:1.

22. The pharmaceutical composition of claim 21, wherein the ratio of flupirtine or a pharmaceutically acceptable salt thereof for controlled-release to flupirtine or a pharmaceutically acceptable salt thereof for immediate-release is about 3:1.

23. The pharmaceutical composition of any one of claims 1 to 22, wherein the composition further comprises at least one exterior phase component.

24. The pharmaceutical composition of claim 23, wherein the exterior phase component is selected from the group consisting of crosscarmellose-Na, microcrystalline cellulose, microdispersed silicon dioxide, magnesium stearate, powder cellulose, calcium hydrogen phosphate dihydrate, lactose, mannitol, starch, and combinations thereof.

25. The pharmaceutical composition of any one of claims 1 to 24, wherein the composition is selected from the group consisting of a tablet, a film tablet, a hard gelatin capsule, a soft gelatin capsule, a pellet, a granulate, a pill, a micro-capsule, and a suppository.

26. A compact comprising flupirtine or a pharmaceutically acceptable salt thereof, wherein the compact has a particle size from about 160 μm to about 800 μm.

27. The compact of claim 26, wherein the compact has a particle size from about 250 μm to about 500 μm.

28. The compact of any one of claims 26 or 27, wherein the compact is coated with a controlled-release component.

29. The compact of claim 28, wherein the controlled-release component comprises a polymer film.

30. The compact of any one of claims 26 to 29, wherein the compact has a bulk volume selected from the group consisting of less than about 5 ml/g, less than about 3 ml/g, less than about 2.5 ml/g, and from about 0.8 ml/g to about 2.5 ml/g.

31. The compact of claim 30, wherein the compact has a bulk volume of less than about 5 ml/g.

32. The compact of claim 30, wherein the compact has a bulk volume of less than about 3 ml/g.

33. The compact of claim 30, wherein the compact has a bulk volume of less than about 2.5 ml/g.

34. The compact of claim 30, wherein the compact has a bulk volume of from about 0.8 ml/g to about 2.5 ml/g.

35. The compact of any one of claims 26 to 34, wherein the compact is spherical or approximately spherical.

36. A kit comprising the pharmaceutical composition of any one of claims 1 to 25.

37. A kit comprising the compact of any one of claims 26 to 35.

38. The kit of any one of claims 36 or 37, wherein the pharmaceutical composition or compact is packaged in the form of individual dosage units.

39. The kit of claim 38, wherein the individual dosage unit is selected from the group consisting of a tablet, a film tablet, a hard gelatin capsule, a soft gelatin capsule, a pellet, a granulate, a pill, a micro-capsule, and a suppository.

40. The kit of any one of claims 36-39, further comprising printed instructions for its use.

41. The kit of claim 40, further comprising a printed matter describing the use of the composition or compact to treat a condition requiring flupirtine therapy, a pre-recorded media device describing the use of the composition or compact to treat a condition requiring flupirtine therapy, or a planner.

42. The kit of claim 41, wherein the printed matter is a book, booklet, brochure, or leaflet.

43. The kit of claim 41, wherein the pre-recorded media device is a DVD, a videotape cassette, a CD-ROM, an audiocassette, or an audio compact disk.

44. A method of flupirtine therapy, the method comprising administering the pharmaceutical composition of any one of claims 1 to 25 to a subject in need thereof.

45. The method of claim 44, wherein the subject is in need of pain relief.

46. The method of claim 45, wherein the subject is in need of acute pain relief.

47. The method of claim 45, wherein the subject is in need of chronic pain relief.

48. The method of any one of claims 44 to 47, wherein the subject is in need of relief from muscle-skeletal pain, post-operative pain, pain after an injury, or pain caused by a tumor.

49. The method of claim 44, wherein the subject is in need of relief of symptoms caused by a disease or disorder treatable by flupirtine administration.

50. The method of claim 49, wherein the disease or disorder is selected from the group consisting of a neurological disorder, a peripheral disorder, episodic ataxia, epilepsy, neuromyotonia, Parkinson's disease, congenital deafness, long QT syndrome, a potassium channelopathy, chronic pain, acute pain, muscle tenseness, apoptotic neuronal cell death, prion disease, Creutzfeldt-Jakob disease, Alzheimer's disease, Tinnitus, overactive bladder, neuropathic pain, neurodegenerative diseases, diabetic neuropathy, diabetic retinopathy, diabetic maculopathy, maculopathy of genetic origin, ocular apoptosis, glaucoma, fibromyalgia, and Batten disease.

51. The method of any one of claims 44 to 50, wherein the subject is a human or a non-human animal.

52. The method of claim 51, wherein the subject is a human.

53. The method of claim 51, wherein the subject is a non-human animal.

54. The method of any one of claims 44 to 53, wherein the composition is administered orally.

55. The method of any one of claims 44 to 53, wherein the composition is administered rectally.

56. The method of any one of claims 44 to 53, wherein the composition is selected from the group consisting of a tablet, a film tablet, a hard gelatin capsule, a soft gelatin capsule, a pellet, a granulate, a pill, a microcapsule, and a suppository.

57. The method of any one of claims 44 to 56, wherein the composition is administered once or twice daily.

58. The method of claim 57, wherein the composition is administered once daily.

59. The method of claim 57, wherein the composition is administered twice daily.

60. A method of preparing compacts comprising flupirtine or a pharmaceutically acceptable salt thereof comprising:
(a) compacting flupirtine or a pharmaceutically acceptable salt thereof to produce compacts of flupirtine or a pharmaceutically acceptable salt thereof, and
(b) selecting compacts with particle sizes from about 160 μm to about 800 μm.

61. The method of claim 60, wherein compacts are selected with particle sizes of about 250 μm to about 500 μm.

62. The method of any one of claims 60 or 61, wherein the method further comprises:
(c) coating at least a portion of the compacts with a controlled-release component, wherein the compacts coated with a controlled-release component are individually coated.

63. The method of claim 62, wherein a separating agent is used to prevent adhesion of compacts.

64. A method of preparing a pharmaceutical composition, wherein the compacts of any one of claims 26 to 35 are further processed into a composition selected from the group consisting of a tablet, a film tablet, a hard gelatin capsule, a soft gelatin capsule, a pellet, a granulate, a pill, a microcapsule, and a suppository.
